(19) United States
(12) Patent Application Publication
(10) Pub. No.: US 2007/0060648 A1
(43) Pub. Date: Mar. 15, 2007

(54) Lipids from methanotrophic bacteria for cholesterol reduction

(76) Inventors: Hanne Muller, Vassbotnen (NO); Anders Skrede, Vassbotnen (NO); Gunnar Kleppe, Vassbotnen (NO)

Correspondence Address: SUGHRUE MION, PLLC, 2100 PENNSYLVANIA AVENUE, N.W., SUITE 800, WASHINGTON, DC, 20037, US

(21) Appl. No.: 10/563,110

(22) PCT Filed: Jul. 2, 2004
(86) PCT No.: PCT/GB04/02866

§ 371 Date: Jun. 19, 2006

(30) Foreign Application Priority Data
Jul. 4, 2003 (GB) 0315783.1

(51) Int. Cl. A61K 31/22; A61K 9/48

(52) U.S. Cl. 514/547; 424/451

(57) ABSTRACT

The present invention provides the use of lipids from methanotrophic bacteria for the manufacture of a composition for oral administration for use in the treatment of animal subjects to reduce plasma cholesterol levels therein or to reduce the ratio of LDL to HDL cholesterol in the plasma thereof. The composition is also useful for increasing plasma docosahexaenoic acid and as immuno-protectant.

This invention relates to the use of microbial cells and extracts and derivatives thereof for the treatment of human or non-human vertebrate animal, especially mammalian, avian or piscine, subjects to modify lipid metabolization, in particular to reduce plasma cholesterol levels therein or to maintain low plasma cholesterol levels therein.

Overly high total plasma cholesterol levels in mammals are associated with increased risk of coronary heart disease. Those particularly at risk include the overweight, smokers, those with a poor diet (e.g. one rich in saturated fats), those who take inadequate exercise, and those suffering from stress. This is not a problem restricted to humans but is also one which occurs with pet animals and farmed animals.

For at risk individuals, as well as those tested and found to have unduly high plasma cholesterol levels, a variety of treatments have been prepared, e.g. changes in diet and habits, increased exercise, etc. However such treatments are not always easy to enforce and there remains a need for treatments which can be effective at reducing plasma cholesterol levels.

In WO 01/60974 and WO 03/016460 there is described a method by which microorganisms may be cultured, with the primary intention of producing a protein-containing material which could be used as a foodstuff or as an additive therefor.

We have now surprisingly found that lipids produced by microbes (e.g. bacteria, yeast or fungi, in particular bacteria) when used as a major or minor part of the lipid intake in an animal's diet, serve to reduce the animal's plasma cholesterol levels. Thus such microbial lipids may be used in a method of treatment to reduce plasma cholesterol, to maintain a reduced plasma cholesterol level, or to reduce the ratio of LDL to HDL cholesterol.

Thus viewed from one aspect the invention provides the use of microbial lipids for the manufacture of a composition, e.g. a pharmaceutical, nutraceutical or foodstuff, for oral administration for use in the treatment of animal subjects to reduce plasma cholesterol, in particular LDL cholesterol, levels therein, or to reduce the ratio of LDL to HDL cholesterol in the plasma thereof, or to increase plasma HDL cholesterol levels.

It has also been surprisingly found that administration of the composition of the invention increases an animal's plasma DHA (docosahexaenoic acid) levels.

In particular, analyses of blood plasma from mink as in Example 1 below surprisingly showed that plasma docosahexaenoic acid (22:6 n-3) increased with increasing level of microbial lipids. The average plasma levels of DHA following feeding of 0%, 17% and 67% microbial lipids (replacing soybean oil) were 5.99, 7.65, and 11.39 mol/100 mol fatty acids. This effect of modification of dietary lipids is highly significant. Neither soybean oil nor the microbial lipids used in the trial contain appreciable amounts of DHA. Thus, higher levels of DHA in the blood plasma of animals fed microbial lipids, compared with soybean oil, were not of dietary origin.

The microbial lipids, particularly the phospholipids and especially phosphatidylethanolamine, thus contribute to higher levels of DHA in blood plasma in the human or animal recipient. Interestingly, the highest level of DHA in the body occurs in phosphatidylethanolamine, the most prominent phospholipid class in the microbial lipid used in the trial. The highest levels of DHA occur in neural tissues, brain and eyes, to a great extent associated with phosphatidylethanolamine. DHA is of great importance in neural tissue, and for reproduction as well as early postnatal life. Fish lipids are the greatest source of DHA in most human diets, and this is an important reason why an intake of fish lipids is so important in human nutrition. Recent published studies show that DHA (and also eicosapentaenic acid (EPA), e.g. 20:5 n-3) is beneficial to cardiovascular health, i.e. improved endothelial function, and lower blood pressure, platelet sensitivity, and serum triglyceride level in humans. The use of the microbial lipids according to the invention is thus beneficial to human and animal health due to increasing the amounts of available DHA in the metabolism.

Thus viewed from a further aspect the invention provides the use of microbial lipids for the manufacture of a composition, e.g. a pharmaceutical, nutraceutical or foodstuff, for oral administration for use in the treatment of animal subjects to increase plasma DHA.

Furthermore, it has been found that juvenile animals especially juvenile fish benefit from an immuno-protective effect brought on by administration of the composition of the invention.

Lipids are the most suitable source of dietary energy in fish feed. It is well known that the n-3 highly unsaturated fatty acids, i.e. 20:5 n-3 and 22:6 n-3, are required to satisfy the requirement for essential fatty acids in fish. The high levels of DHA in neural tissue may be particularly importance in the feeding of fish larvae. In fish feed, fish oil is the main dietary source of these fatty acids. Fish oil is at present a limited resource, and insufficient fish oil supply is expected to limit expansion in aquaculture in the future. There is evidence that fish larvae have limited capacity to synthesise phospholipids. The use of the microbial lipids according to the invention, by virtue of their high contents of phospholipids, in particular phosphatidylethanolamine, is thus beneficial to fish health and survival, especially in the early (eg first feeding to juvenile) stages of life. This is also particularly due to the effect of increasing levels of DHA available in the metabolism.

Thus from a further aspect, the invention provides the use of microbial lipids for the manufacture of a composition, e.g. a pharmaceutical, nutraceutical or foodstuff, for oral administration for use in the treatment of animal subjects as an immunoprotectant.

The word animal as used herein is used to refer to humans and non-humans, particularly vertebrates, e.g. mammals, birds and fish, but also, less preferably shellfish. Use of the invention in relation to humans and other mammals as well as gallinaceous birds (e.g. domestic fowl) and farmed fish (e.g. salmon and trout) is particularly preferred. Use in relation to humans, non-human mammals and domestic fowl is especially preferred. Particularly preferred is use in relation to juvenile animals, especially juvenile fish.

Viewed from a further aspect the invention provides a method of treatment of an animal subject to reduce plasma cholesterol levels therein, to maintain a reduced plasma cholesterol level therein or to reduce the ratio of LDL to HDL cholesterol in the plasma thereof, said method comprising orally administering to said subject, e.g. as part of its dietary intake, an effective amount of a microbial lipid.

By a microbial lipid is meant herein a lipid produced by a microbe, e.g. a bacteria, yeast or fungus. Such lipids will generally be produced so as to constitute part of the microbe's internal or external membranes. Typically such lipids comprise phospholipids, e.g. phosphatidylglycerols, phosphatidylethanolamines, phosphatidylcholines, and cardiolipins. In the case of bacteria, the preferred source of the microbial lipids used according to the invention, the lipids in the microbes are majoratively phospholipids (i.e. the head group to which the fatty acid side chains are attached includes a phosphorus atom). The use of bacteria which have a high phospholipid content and in particular a high phosphatidylethanolamine content (relative to total lipid content, e.g. a phospholipid content of at least 30% wt., preferably a phosphatidylethanolamine content of at least 30% wt. relative to total lipid content) is especially preferred, e.g. Gram-negative bacteria and in particular bacteria having membranes for nutrient gas fixation, particularly methanotrophic bacteria, and especially methylococcus bacteria. The use of bacteria in which phospholipids, and especially phosphatidylethanolamines, constitute at least 60% wt of the total lipid contact is especially preferred.

Quite surprisingly such microbial phospholipids have a marked cholesterol reducing effect despite the fact that the fatty acid side chains are predominantly saturated or monounsaturated (i.e. they are fatty acids of a type which would be expected to increase plasma cholesterol).

Interestingly, plasma triacylglycerol levels may also be reduced by administration of microbial lipids in accordance with the present invention and in further aspects of the invention the reduction in triacylglycerols rather than or as well as an effect on cholesterol may be the aim of the claimed products or methods.

The fatty acid side chains in such lipids are generally C₁₄ to C₂₂ fatty acids, particularly C₁₆ fatty acids and in a preferred embodiment these are predominantly (e.g. >80% wt.) saturated or monounsaturated fatty acids, especially C_{16:0} and C_{16:1} fatty acids. The microbial lipid used according to the invention preferably contains phosphatidylethanolamines as the major lipid constituent, e.g. at least 50% wt., more preferably at least 65% wt.

The microbial lipids used according to the invention may if desired be separated from the other components of the microbes before use, e.g. from proteins, nucleic acids, etc. However, since the microbial lipids may be used as part of the dietary intake of the animal, i.e. to meet part of its nutritional needs, such lipid separation is not essential since the other components may also contribute to the animal's nutritional needs.

In general, it is preferred that the microbial lipid be used in the form of a lipid extract from a microbial biomass, e.g. a culture, prepared for example as described in WO 01/60974 and WO 03/016460, the contents of which are hereby incorporated by reference. The homogenization step referred to in WO 01/60974 may be omitted and the biomass from the reactor may be subjected to a conventional lipid extraction technique, e.g. supercritical extraction or solvent extraction. Preferably such extraction will involve use of a polar organic solvent or solvent mixture, e.g. a mixture of an alcohol and a halocarbon, especially a methanol/chloroform mixture, particularly a 1:2 v/v methanol/chloroform mixture. Such an extraction process forms a further aspect of the present invention.

Microbial lipid extracts are themselves novel and form a further aspect of the invention. They are chemically distinct from plant derived lipids in terms of their fatty acid profile as they are substantially free from polyunsaturated fatty acids. As a result they have increased storage stability. Viewed from this aspect the invention thus provides a microbial lipid, preferably at least 80% wt. pure, especially at least 90% wt. pure, particularly at least 95% wt. pure, e.g. >99% wt. pure, and preferably in a quantity of at least 10 g, especially at least 50 g, more especially at least 100 g.

Following extraction from the microbial biomass, the lipid extract may if desired be purified or separated into lipid fractions (e.g. chromatographically). The phospholipid (-containing) fractions, and in particular the phosphatidylethanolamine (-containing) fractions, especially the fraction(s) containing phosphatidylethanolamines with C_{16:0} and/or C_{16:1} fatty acid side chains, are especially preferred for use according to the invention.

Viewed from a further aspect the invention provides a microbe-derived phospholipid, especially a phosphatidylethanolamine, and particularly a phosphatidylethanolamine with C_{16:0} and/or C_{16:1} fatty acid side chains, preferably at least 80% wt. pure, especially at least 90% wt. pure, particularly at least 95% wt. pure, e.g. >99% wt. pure, and preferably in a quantity of at least 10 g, especially at least 50 g, more especially at least 100 g, e.g. for use in medicine.

Such microbial lipid extracts may be used as ingredients in foodstuffs, e.g. as a total or partial replacement for fats contained therein, or as an additional lipid component. Typical such foodstuffs include margarines and other spreads, mayonnaise and other salad dressings, yogurts, creams (including non-dairy creams etc.), cheeses and cooking oils and fats. Such foodstuffs form a further aspect of the invention.

Alternatively, the microbially derived lipids may be administered in pharmaceutical or nutraceutical form, e.g. formulated as solutions, suspensions, emulsions micro-emulsions, vesicles or micro-encapsulated forms, or in capsules, etc. Conventional pharmaceutical carriers and excipients, such as stabilizers, emulsifiers etc. and conventional formulation techniques may be used. Where the product is in dosage unit form, the dose unit preferably contains 100 to 1500 mg, especially 300 to 700 mg, particularly 400 to 600 mg. Formulation in capsule form is especially preferred, e.g. using gelatin capsule cases. A daily dose would typically be 0.02 to 0.5 g/kg bodyweight, preferably 0.05 to 0.25 g/kg bodyweight.

Thus viewed from a further aspect the invention provides a foodstuff containing a microbial lipid extract (preferably providing at least 5% wt. of the total lipid content of the foodstuff, more preferably at least 10% wt, especially at least 25% wt.) and a further nutrient component, preferably of non-microbial origin.

Viewed from a still further aspect the invention also provides a pharmaceutical or nutraceutical composition comprising a microbial lipid extract together with a pharmaceutically acceptable carrier or excipient.

The animal recipient of the microbial lipids according to the invention will preferably be a mammal or bird found to have elevated plasma cholesterol levels or considered to be at risk of elevated plasma cholesterol levels, e.g. due to its weight, diet, habits or living conditions. Such animals include in particular humans and fish; however the invention is also applicable to pet animals (e.g. dogs, cats, rabbits, guinea pigs, etc.) and to farm animals, (e.g. poultry (for example chickens, ducks, geese and turkeys), pigs, cows, goats and sheep). The microbial lipids preferably constitute from 1 to 99% wt. of the animal's dietary lipid intake, more especially 10 to 90% wt. particularly 20 to 80% wt., more particularly 30 to 70% wt. In general, it is preferred that the animal's diet includes further lipids of non-microbial origin, e.g. fish oils or plant oils, to ensure the necessary intake of polyunsaturated fatty acids and C₁₈ fatty acids. Soy bean oil, rape seed oil, sunflower oil, maize oil, evening primrose oil and fish oils are especially preferred for use in this regard.

The use of the microbial lipids in the feed for food animals (e.g. chicken etc.) may also lower the cholesterol in the meat or eggs (avian eggs that is) that are produced from or by such animals for human consumption and such reduced cholesterol food products provide a still further aspect of the present invention. Desirably the animal is fed the microbial lipids for a period of at least one week, preferably at least two weeks before the reduced cholesterol food product (e.g. meat or poultry eggs) is harvested.

The microbial lipids (or a microbe-based product such as a homogenizate etc.) may be administered alone; however more generally they will be formulated together with other nutritionally useful materials, e.g. meat, plant oils, fish oils, carbohydrates, flavours, vitamins, minerals, etc. Such a formulated product, since it has a therapeutically or prophylactically desired effect as well as a nutritional effect will generally be referred to as a functional food or as a nutraceutical composition. It is preferred that the microbial lipid constitute from 1 to 99% wt. of such a nutraceutical composition, more preferably 2 to 50% wt., especially 5 to 20% wt. If the nutraceutical composition, as is preferred, is in the form of a total feed (i.e. one which can supply the entire nutritional needs of the animal in terms of protein, carbohydrate and lipids), then it may be given to the animal at frequencies and in quantities routine for food for the size, age, gender and species of the animal in question.

The microbe source of the microbial lipid is preferably a bacterium or a mixture of bacteria cultured using methane as the carbon source, e.g. a Methylococcus species such as M. capsulatus, optionally together with Ralstonia (formerly Alcaligenes) and/or Bacillus and/or Aneurinibacillus species, such as Ralstonia sp, Aneurinibacillus sp and B. agri. Such strains are available from Norferm Danmark A/S, Stenhuggervej 22, DK-5230 Odense, Denmark. The microbes might be administered as live cultures; however more normally the microbe will be killed before administration (e.g. as a natural consequence of a homogenization or sterilization step, or before lipid extraction).

Quite surprisingly, if microbial lipids are extracted from the microbial biomass, the residual material (which contains proteins, nucleic acids, carbohydrates, etc) is of enhanced digestibility relative to the biomass from which the lipid has not been extracted. The lipid-depleted biomass, and lipid-depleted biomass derivatives (e.g. homogenizates) their production by lipid extraction therefrom and their use in or as feedstuffs or feedstuff additives all form further aspects of the present invention.

The invention will now be described further with reference to the following non-limiting Examples.

### EXAMPLE 1

Mink

The effects on plasma cholesterol in mink (Mustela vison) of three different high lipid diets (56 E % from lipids, i.e. with lipids providing 56% of dietary energy input) with lipids extracted using methanol and chloroform from BPM (a bacterial biomass) replacing 0%, 17% and 67% of those in soybean oil, were studied. BPM is produced by culturing Methylococcus capsulatus, Rastonia sp., Brevibacillus agri and Aneurinibacillus sp. using natural gas (99% methane), ammonia and mineral salts as described in WO 01/60974 and WO 03/016460. Phospholipids are the main lipid component in BPM, consisting mainly of phosphatidylethanolamines, together with some phosphatidylglycerols, with predominantly 16:0 and 16:1 fatty acids and no polyunsaturated fatty acids.

The 0%, 17% and 67% bacterial lipid diet compositions were as set out in Table 1 below.

TABLE 1

| Ingredient | 0% | 17% | 67% |
|---|---|---|---|
| Corn starch | 62.9 | 62.9 | 62.9 |
| Coalfish fillet | 651.4 | 651.4 | 651.4 |
| Soybean oil | 91.4 | 61 | 15.3 |
| Bacterial lipids | — | 30.5 | 76.2 |
| Sunflower oil | 2.29 | 2.29 | 2.29 |
| Vitamin/mineral mix (100 | 0.84 | 0.84 | 0.84 |
| kCal/100 g) | | | |
| BHT (100 mg/kg) | 0.08 | 0.08 | 0.08 |
| Calcium phosphate | 1.63 | 1.63 | 1.63 |
| Calcium carbonate | 1.85 | 1.85 | 1.85 |
| Water | 187.7 | 187.7 | 187.7 |

The figures in columns are in g/kg.

18 growing mink were fed one of the three diets during a 25-day period in a parallel group design. The mink had their main part of the plasma cholesterol in the HDL fraction. Total cholesterol, LDL cholesterol, HDL cholesterol, VLDL cholesterol and the LDL/HDL cholesterol ratio were significantly lower by 34.5%, 49.2%, 28.8%, 29.7%, and 26.8%, respectively, when the mink consumed diets with 67% lipids from BPM and 33% lipids from soybean oil as compared to diets containing 100% lipids from soybean oil.

These reductions are especially significant since soybean oil itself is known to have a plasma cholesterol lowering effect.

The plasma concentrations of triacylglycerols (i.e. triglycerides) were also measured and were lower by 31.0% when the mink consumed diets with 67% lipids from BPM and 33% lipids from soybean oil as compared to diets containing 100% lipids from soybean oil.

### EXAMPLE 2

Pigs

Pigs were fed BPM as 0, 5, 10 and 15% wt of their diet. BPM contains about 10% wt microbial lipids and thus the highest BPM content diet provided about 25% wt ototal dietary lipids. Plasma from the pigs was analyzed for cholesterol content and, while the total cholesterol content was not significantly altered, the proportion of the cholesterol which was HDL cholesterol, ie the cardioprotective form, was increased. The plasma HDL cholesterol levels four the four diets were respectively 1.017, 1.077, 1.073 and 1.227 mol/L.

1-17. (canceled)

18. A method for reducing plasma cholesterol of an animal or maintaining a reduced cholesterol level in an animal or reducing LDL:HDL cholesterol ratio in plasma of an animal comprising orally administering to an animal in need thereof, an oral dosage composition comprising an effective amount of microbial lipids.

19. A method for increasing plasma docosahexanenoic acid (DHA) in an animal comprising orally administering to an animal in need thereof, an oral dosage composition comprising microbial lipids.

20. A method for achieving an immuno-protectant effect in an animal comprising orally administering to an animal in need thereof, an oral dosage composition comprising microbial lipids.

21. The method as claimed in claim 18, wherein said animal is a human.

22. The method as claimed in claim 19, wherein said animal is a human.

23. The method as claimed in claim 20, wherein said animal is a human.

24. The method as claimed claim 18, wherein said animal is a fish.

25. The method as claimed claim 19, wherein said animal is a fish.

26. The method as claimed claim 20, wherein said animal is a fish.

27. The method as claimed in claim 24, wherein said fish is a juvenile fish.

28. The method as claimed in claim 25, wherein said fish is a juvenile fish.

29. The method as claimed in claim 26, wherein said fish is a juvenile fish.

30. The method as claimed in claim 18, wherein said microbial lipids are methanotrophic bacterial lipids.

31. The method as claimed in claim 19, wherein said microbial lipids are methanotrophic bacterial lipids.

32. The method as claimed in claim 20, wherein said microbial lipids are methanotrophic bacterial lipids.

33. The method as claimed in claim 18, wherein said composition is an encapsulated composition.

34. The method as claimed in claim 19, wherein said composition is an encapsulated composition.

35. The method as claimed in claim 20, wherein said composition is an encapsulated composition.

36. A composition comprising at least 80% wt. microbial lipid, and comprising at least 10 g of said microbial lipid.

37. The composition as claimed in claim 36, wherein said lipid is phosphatidylethanolamine.

38. The composition as claimed in claim 37, wherein said phosphatidylethanolamine is a phosphatidylethanolamine having C_{16:0} and/or C_{16:1} fatty acid side chains.

39. A foodstuff comprising a microbial lipid extract and a nutrient component.

40. The foodstuff as claimed in claim 39, wherein said nutrient component is of non-microbial origin.

41. A pharmaceutical or nutraceutical composition comprising a microbial lipid extract together with a pharmaceutically acceptable carrier or excipient.

42. A food product harvested from an animal fed with a microbial lipid.

43. The food product as claimed in claim 42, wherein said food product is an avian egg.
